(19) United States
(12) Patent Application Publication
(10) Pub. No.: US 2004/0043477 A1
(43) Pub. Date: Mar. 4, 2004

(54) Biosensor and method of production thereof

(76) Inventor: Peter Urs Schibli, Solothurn (CH)

Correspondence Address: Douglas L Christensen, Patterson Thuente Skaar & Christensen, 4800 IDS Center, 80 South eight Street, Minneapolis, MN, 55402, US

(21) Appl. No.: 10/312,939

(22) PCT Filed: Jun. 25, 2001
(86) PCT No.: PCT/EP01/07164

(30) Foreign Application Priority Data
Jun. 30, 2000 (EP) 00113906.2

(51) Int. Cl. C12M 1/34

(52) U.S. Cl. 435/287.1

(57) ABSTRACT

The invention relates to a biosensor for the determination of substances in bodily fluids, in particular, in blood, comprising a two-piece support plate, whereby the one piece of the support plate represents an upper piece and the other piece represents a lower piece, with an intermediate layer lying between the upper and lower piece in which an aperture is formed. The upper piece, lower piece and aperture form a capillary channel, running from a cover opening formed at the edge of the biosensor to an air hole formed in the upper piece or lower piece. Electrodes are provided, which together with an enzyme-containing substance permit an electrochemical measurement of substances found in body fluids. The upper piece and lower piece each support at least one electrode in the region of the capillary channel, which oppose each other in pairs and are arranged such that both electrode pairs form measuring regions lying within the capillary channel, whereby at least one of the electrodes of each electrode pair is treated with an enzyme-containing substance.

The invention relates to a biosensor for determining substances in body liquids as well as to a method of producing such biosensor.

The determination of substances in body liquids using biosensors is known. For example, glucose may be determined in urea or in blood using the glucose oxidase enzyme (cf. Carlson, “Kurzes Lehrbuch der Biochemie für Mediziner and Naturwissenschaftler”, 11^{th} edition, p. 189). To do so the glucose contained in e.g. a drop of blood is oxidated to gluconolactone by glucose oxidase. The electrons released thereby reduce the glucose oxidase enzyme, which, in turn, transfers electrons to a mediator, e. g. ferrocene, with the enzyme being oxidized in turn. The electrons may be transferred to an electrode by the mediator, so that a microcurrent flows when a voltage is applied. Said current may be used as a measure for the glucose content of the blood sample. This enzymatic reaction is, thus, electrochemically sensed in a biosensor by measuring a current between two electrodes after applying a voltage. Biosensors operating on this principle are known, for example, from U.S. Pat. No. 5,264,130, U.S. Pat. No. 5,264,106 or from EP-A O 0 359 831.

The latter document, on which the preamble of claim 1 is based, discloses a biosensor composed of a two-part base plate, wherein one part forms the upper part and the other part forms the lower part, and an intermediate layer, in which a slit is located, is provided between said upper and lower parts. Said slit terminates, on the one hand, in an air vent and, on the other hand, in an opening in the edge of the biosensor, through which body liquid is supplied. An electrode assembly and an enzyme-containing substance allowing the aforementioned electrochemical measurement of substances are provided in the region of the slit on the lower part.

The object underlying the invention is to improve a biosensor of the aforementioned type with regard to its detection properties and to provide a method of producing such biosensor.

According to the invention, in a biosensor for determining substances in body liquids, said biosensor comprising a two-part base plate, wherein one part of the base plate forms an upper part and the other part forms a lower part, and comprising an intermediate layer, in which a slit is formed, between said upper part and said lower part, with said upper part, said lower part and said slit forming a capillary channel extending from a supply inlet formed at the edge of the biosensor up to an air vent formed in the upper or the lower part, and electrodes are provided which, together with an enzyme-containing substance, allow an electrochemical measurement of substances contained in body liquids, this object is achieved in that the upper part and the lower part each carry at least one electrode in the region of the capillary channel, which electrodes are located opposite each other in pairs and are arranged such that each pair of electrodes forms a measuring region located in the capillary channel, wherein an enzyme-containing substance is applied on at least one electrode of at least one pair of electrodes.

It is essential to the concept of the invention that the electrodes should not be located exclusively on the lower part of a multipart sensor. Instead, the surface area available on the upper part, according to the invention, is also equipped with electrodes such that the electrodes are arranged in pairs lying opposite each other.

This biosensor has the advantage over the prior art that the effective electrode surface area is strongly increased by the electrodes being arranged in pairs lying opposite each other. This increases the usable signal which depends directly on the electrode surface area in an electrocemical measurement. Thus, the signal/noise ratio is markedly improved, which lowers the detection limits attainable with said biosensor for the substances to be sensed. The improved signal/noise ratio allows analyzes to be carried out using smaller volumes of body liquid. For example, the minimum volume of blood required for a measurement of blood sugar levels is considerably smaller, which is more pleasant for the patient, because it causes less pain to obtain a drop of blood.

Arranging the electrodes in pairs lying opposite each other further has the advantage that virtually any number of pairs of electrodes may be provided in the capillary channel in series. This enables detection of not just one, but actually of several substances in a body liquid sample.

The arrangement of the electrodes in pairs lying opposite each other further results in the advantage that the body liquid to be analyzed passes through exactly between all of said electrode pairs and does not flow successively over serially arranged electrodes associated with each other in pairs. Thus, the current picked up by the electrodes flows transversely of the flow direction of the body liquid, which is advantageous with a view to precise measurements, since the measurement signal shows a more step-like response when the body liquid enters into the space between each pair of electrodes, than if said liquid flowed over serially arranged electrodes. Thus, the minimum volume of body liquid required for analysis is further reduced.

Particular advantages in manufacture are achieved if a base plate is used which is folded along a folding line, i. e. if the upper and lower parts are parts of an integral base plate. This simplifies application and contacting of the electrodes, because it may be effected, prior to folding, in one operation and on one surface of a member. Moreover, by folding the base plate along a folding line which separates the upper and lower parts, particularly good registration of the oppositely arranged pairs of electrodes is ensured, thus obviating the need for registering and adjusting structures. Also, the application of the enzyme-containing substance to the base plate prior to folding is easier. Further, this concept allows relatively easy application of the intermediate layer comprising the slit on the base plate, for example by a screen printing method.

This further embodiment allows all electrodes to be contacted via contacts located either on the upper or on the lower part, since the electrodes are applicable to the base plate in one operation prior to folding. For this purpose, the upper or the lower part is preferably provided with a section protruding in the folded state and carrying said contacts, which section is adapted for insertion into an evaluation device, which then establishes an electrical connection to the electrodes via said contacts.

As the material for the upper and the lower part or for the base plate, any material is suitable which is, on the one hand, sufficiently inert with regard to the body liquid to be analyzed and to the enzyme paste to avoid cross-sensitivity and errors of measurement, and which, on the other hand, enables a capillary effect in the capillary channel due to its wetting characteristics. Thin sheets are particularly preferable, with Kapton or polyester being advantageous from the point of view of costs. For rigidification, a stabilizing support layer may be provided under the sheet of the lower part, thus enabling selection of particularly thin sheets.

In principle, the supply inlet for introducing the body liquid to be analyzed into the capillary channel may be arranged at any desired location. The channel merely needs to extend up to an edge of the biosensor, for example up to the edge of the base plate. However, it is particularly preferred if a hole is provided in the region of the folding line, up to which hole the slit in the intermediate layer extends. After folding of the base plate, said hole then forms the supply inlet. A biosensor is particularly convenient for a patient who wishes to introduce a drop of blood from one finger into the supply inlet, wherein the supply inlet provided on the edge formed by the folding line is a curved recess, for example, having the profile of a fingertip. The patient, having punctured his finger with a needle as usually done in order to obtain a drop of blood, then simply has to place his finger in the recess at the edge of the biosensor.

When applying such drop of blood from a fingertip to a supply inlet, it is usually a problem for the patient to know whether he has actually managed to introduce his drop of blood into the supply inlet. The patient is no longer faced with this problem if the recess at the edge of the biosensor, in a further preferred embodiment of the biosensor of the invention, is provided, in the lower part, with an edge face which is perpendicularly to the top surface of the biosensor, and, in the upper part, with an edge face protruding and, in particular, being obliquely arranged relative to the top surface. Particularly preferably, the blunt edge of the obliquely arranged edge face is generally in alignment with the perpendicularly edge face of the recess in the lower part. A protruding oblique surface designed in this manner functions, in a manner of speaking, as a protection against being used upside down. In the design comprising the base plate, the hole may then be quite intentionally designed in an asymmetrical manner, so that the edge of the hole lying in the lower part is not arranged in exact alignment above the edge of the hole in the upper part upon being folded.

Using such protection, a patient need no longer place the finger on a narrow edge face extending perpendicularly to the flat biosensor, but the protruding, in particular oblique, edge of the recess at the upper part enables him to ascertain whether the drop of blood actually hits the supply inlet to the capillary channel. Said protection is particularly effective if the oblique surface extends at an angle of about 30 to 40° to the top surface.

This concept is very advantageous, in particular, bearing in mind that such biosensors are also used by elderly patients.

Due to the shape of the slit, the capillary channel may be given virtually any design, as long as it is ensured that a capillary effect is produced, i. e. that body liquid introduced at the supply inlet is actually transported by capillary forces along the capillary channel. In a further embodiment of the invention, the design of the capillary channel as a non-straight capillary channel controls the speed at which said body liquid is transported within said channel. If the capillary channel widens, i. e. if the width of the slit in the intermediate layer and thus the width of the capillary channel as viewed away from the supply inlet increases, the body liquid will be rapidly transported away from the supply inlet. If the width of the slit and, thus, the cross-section of the capillary channel is designed to decrease, suction will be slow. Thus, the speed at which the biosensor responds may be designed according to the particular application.

The intermediate layer serves to form the capillary channel together with the upper and the lower part. Like the material for the upper and the lower part, said layer is, therefore, inert to the body liquid to be analyzed and designed such that it does not lead to errors of measurement. Further, it does not harm the enzymes used.

For reasons of manufacture, a two-layer intermediate layer is particularly preferred which consists of a varnish layer of suitable thickness applied on the lower part, said varnish layer being bonded to the upper part by an adhesive. The adhesive is designed such that its curing process will not harm the enzymes. This may be achieved, in particular, by adhesives curing under humidity or activated by pressure.

The communication with the patient using the biosensor is important. Therefore, in a further embodiment of the biosensor, a light guiding element is arranged on both edges of the upper or lower part comprising the protruding section, which light guiding element can be illuminated upon insertion into the evaluation device. For example, depening on the light incident through the evaluation device, the light guiding element emits light of a certain color, e. g. red or green light. Said light guiding element may be obtained by imprinting the edge of the upper and/or lower part such that a light-guiding radiation channel is produced in the form of a guiding line at the edge of the sensor. Thus, the evaluation device will contain a red or a green light source, for example, an LED. According to the light emitted by said LED, the patient will know what to do.

In principle, any enzymes which enable electrochemical measurement may be used in the enzyme-containing substance. It is particularly preferred if the enzyme is selected from the following group: lactate oxidase, glucose oxidase, cholesterase, uricase, xanthine oxidase, peroxidase, urease, aminotransferase, cholesterol oxidase, aminooxidase, glutamate oxidase, creatinine oxidase, creatinine aminohydrolase and dehydrogenases. Of course, different enzyme-containing substances may be used for individual pairs of electrodes. Thus, the biosensor may measure several different substances in a body liquid sample. It is certainly also possible to use a pair of electrodes as a reference pair of electrodes in order to obtain a zero value, as is known from the state of the art.

The biosensor according to the invention is produced by:

a) producing a base plate, which comprises two parts connected with each other and foldable along a folding line, one of said parts of the base plate forming an upper part and the other of said parts forming a lower part of the biosensor,

b) forming a through hole in the base plate,

c) applying a conducting structure comprising electrodes, which are connected to contacts on the upper or the lower part via conductors, wherein two electrodes are arranged on the upper part and two electrodes are arranged on the lower part, and

d) applying an intermediate layer on the upper or the lower part, said intermediate layer being provided with a slit which extends up to the hole and is arranged above the electrodes of the upper or the lower part.

This method allows production of the conducting structure and of the intermediate layer by a screen printing method. Screen printing methods are, on the one hand, quite inexpensive and, on the other hand, allow exact positioning of the electrodes or of the structures of the intermediate layer. In the embodiment of the sensor which is folded along the folding line, said folding simultaneously ensures exact registration of the pairs of electrodes arranged opposite each other.

In biosensors there is usually the problem that storage of the enzyme-containing substances upon their preparation is possible only for a short time. Moreover, in many cases, special storage conditions, e.g. low temperatures, must be met. The method of production according to the invention allows to suspend production of the biosensor once the intermediate layer has been applied. If an adhesive layer is selected as the intermediate layer, it may be covered with a protective sheet. Up to this point, the biosensor can be produced in very large quantities and stored for as long as desired. In order to finally produce the quantity of biosensor intended for consumption in the very near future, one only has to remove the protective sheet and apply the enzyme-containing substance. If use is made of the design comprising the two-layer intermediate layer, the protective sheet may even be dispensed with if an adhesive activated by pressure is used. Upon folding, the biosensor will be ready for use.

Particularly preferably, the production of a biosensor comprising a supply inlet in the region of the folding line may be effected by punching a preferably lens-shaped hole in the region of the folding line. Said hole should be formed such that, upon folding, the edge of the hole located in the lower part comes to rest upon the edge of the hole located in the upper part. Thus, said hole needs to be essentially symmetrical. Of course, the slit in the intermediate layer needs to be provided such that it extends up to the hole.

When punching said hole, the edge faces may be formed as mentioned above, i.e. the edge face of the hole located in the lower part will extend perpendicularly to the top surface and the edge face located in the upper part will extend obliquely to the top surface.

Alternatively, the supply inlet of the capillary channel is located in a stepped edge. The hole is then to be punched asymmetrically. This embodiment also facilitates application of the drop of blood by the patient. It is easier to produce than that comprising the oblique edge face of the lower part, while offering similar advantages in use.

The biosensor produced in this manner is suitable, in particular, for determining blood levels, in particular of blood sugar, urea, lactate, cholesterol, vitamins, troponin, and myoglobin.

Further advantageous embodiments of the invention are addressed in the subclaims.

In the following, the invention will be explained in more detail by means of embodiment examples with reference to the drawings whose entire disclosure is essential to the invention and wherein:

FIG. 1 shows an exploded view of a biosensor;

FIG. 2 shows a representation of a base plate of a biosensor prior to folding;

FIG. 3 shows a base plate of a biosensor during folding, and

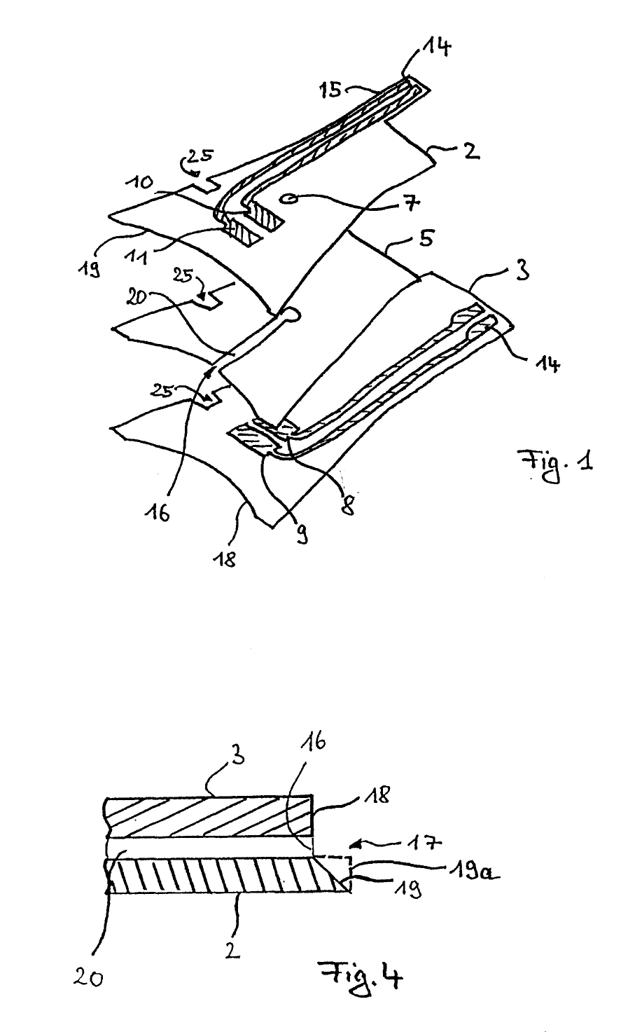

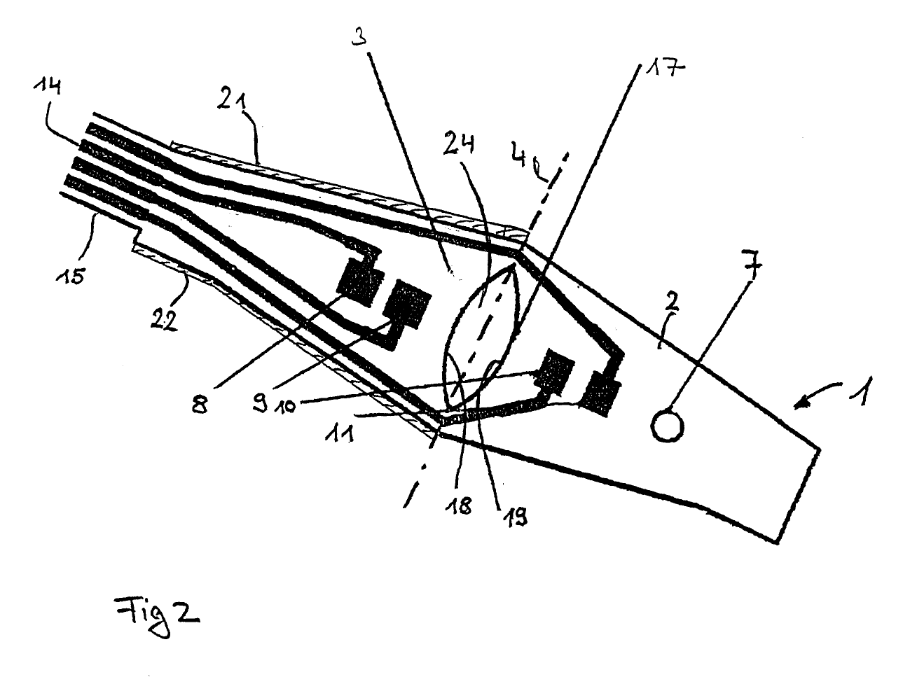

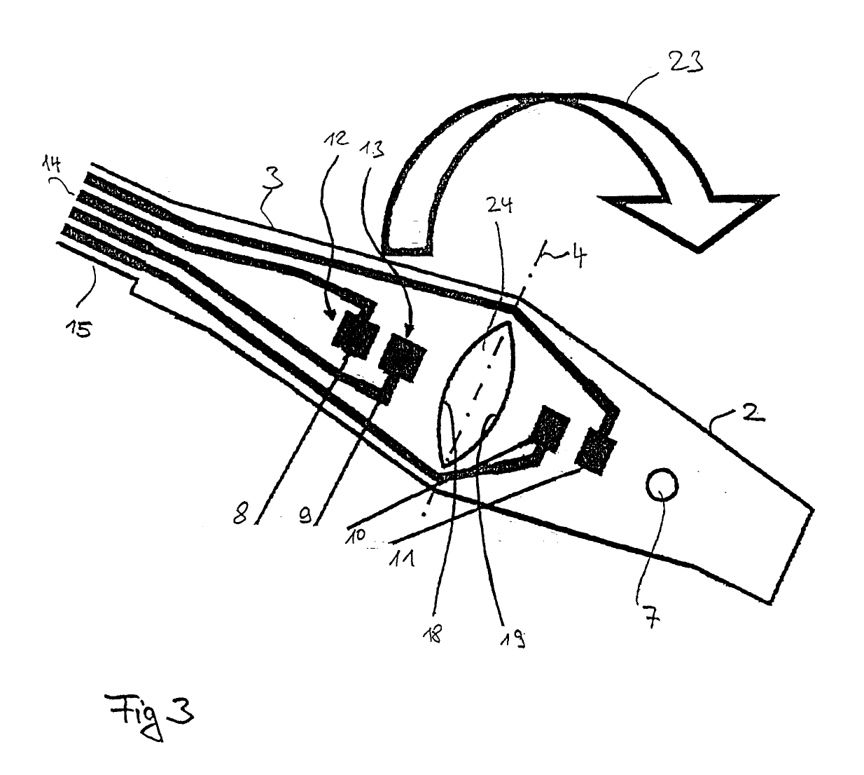

FIG. 4 shows an enlarged sectional view of a supply inlet of the biosensor of FIGS. 1 to 3.

FIG. 1 shows an exploded view of a biosensor. This biosensor is designed for insertion into an evaluation device and has a corresponding section 15 comprising contacts 14 which are electrically contacted upon insertion into the slit of an evaluation device. The biosensor consists of an upper part 2 and of a lower part 3, both made from plastic sheet having a thickness of 10 μm-200 μm. An intermediate layer 5 is arranged between the upper part 2 and the lower part 3 and connects the upper and lower parts with each other. The upper part 2 is provided with an air vent 7 whose function will be explained later. On its surface lying adjacent the intermediate layer 5, the upper part 2 has two electrodes 10 and 11 which, however, are shown on the side facing away from the intermediate layer 5 in FIG. 1 for better illustration. The lower part 2 is provided with similar electrodes 8, 9. The upper part 2 and the lower part 3 comprise the intermediate layer 5 therebetween in such manner that the electrode 10 lies directly opposite the electrode 8 and the electrode 9 lies directly opposite the electrode 11.

The intermediate layer 5 has a slit 6 formed therein, which extends via the pairs of electrodes 9, 11 and 8, 10 up to the air vent 7. Said slit 6 starts at an edge of the biosensor, as will be explained later. Together with the upper part 2 and the lower part 3, the slit 6 forms a capillary channel 20 which serves to transport body liquid for analysis. The slit terminates in a supply inlet 16 which is located at an edge of the biosensor. The intermediate layer is made up of two layers and consists of a varnish layer applied on the lower part and an adhesive layer applied thereon for fixing the upper part.

The electrodes 8, 9 are connected to contacts 14 on the lower part 3, while the electrodes 10 and 11 are connected to identical contacts 14 on the upper part 2.

The lower part 3, the intermediate layer 5 and the upper part 2 are joined so as to align, for which purpose a registering structure 25 is provided in each of said three parts. Said structures may be, for example, grooves or notches which are registered with each other. Once they have been aligned, the electrodes 11, 9 and 8, 10 of the pairs of electrodes will be located exactly opposite each other in the capillary channel 20.

The edge of the biosensor is provided with a recess 17 in the region around the supply inlet 16, said recess 17 enabling easy application of the body liquid to be analyzed, for example blood from a patient's fingertip. The edge faces 18 and 19 of the lower part 3 or of the upper part 2 are designed as shown in FIG. 4. FIG. 4 shows a partial section through the biosensor along the capillary channel 20. The edge face 18 of the recess 17 in the lower part 2 extends perpendicularly to the top surface of the lower part 3. In contrast thereto, the edge face 19 of the recess 17 in the upper part 2 extends obliquely relative to the top surface of the upper part 2. In this case, the edge faces 18 and 19 are arranged relative to each other in such manner that the edge face 19 protrudes over the borders of the edge of the lower part 3. The angle occupied by the edge face 19 relative to the top surface of the lower part 3 is between 30° and 40°. Said obliquely extending edge face serves for rotation protection and centering, assisting the patient in introducing into the supply inlet 16 a drop of blood present, for example, on the fingertip. The edge faces 18 and 19 of the punched hole 24 are shown in FIG. 4. Alternatively, the edges 18, 19 may be designed such that, while they both occupy the same angle relative to the top surface, one of said edge faces protrudes slightly, as indicated in broken lines for edge face 19a in FIG. 4.

FIG. 2 shows an alternative embodiment of the biosensor of FIG. 1. In this embodiment, the upper and lower parts 2, 3, which are provided as separate parts in FIG. 1, are connected with each other along a folding line 4, i. e. they are integral parts of a base plate 1. Said base plate 1 is imprinted with electrodes 8 to 11 as well as with the contacts 14 and respective conductors. As in the above embodiment according to FIG. 1, the intermediate layer 5 is also applied on the upper part 2 or the lower part 3 by a printing method. Next, the base plate 1 is folded along the folding line 4. If required, a corresponding groove is formed in the surface of the base plate 1 opposite the printed side, in the region of the folding line 4, in order to ensure easy folding even for a thicker base plate 1. How said folding is effected will be explained hereinbelow with reference to FIG. 3.

The capillary channel now terminates in the edge of the biosensor, at which the folding line 4 is located. Accordingly, the recess 17 is provided as a lens-shaped punched hole 24 in the base plate.

The capillary channel formed by the upper part 2, the lower part 3 and the slit 6 may be provided as a non-straight or oblique capillary. For this purpose, the capillary channel widens or tapers from the supply inlet 16 toward the air vent 7, which results in different flow characteristics. If the capillary channel tapers from the supply inlet 16 toward the air vent 7, a slower flow characteristic of the body liquid is achieved. If the capillary channel 20 widens from the supply inlet 16 toward the air vent 7, a faster flow characteristic is obtained.

The air vent 7 is essential, since only then the capillary forces cause sufficiently rapid suction of a body liquid into the capillary channel 20.

An enzyme-containing substance is applied on one of the electrodes 8 or 11 as well as one of the electrodes 9 or 10 of the pairs of electrodes 8, 11 and 9, 10. In doing so, one of the following enzymes may be used: lactate oxidase, glucose oxidase, cholesterase, uricase, xanthine oxidase, peroxidase, urease, aminotransferase, cholesterol oxidase, aminooxidasen, glutamate oxidase, creatinine oxidase, creatinine aminohydrolase and dehydrogenases.

The operational principle of the measurement will be discussed later.

The sensor of FIG. 2 is now produced as follows. First, the base plate 1 is produced, for example, by punching it out of a larger plate. Suitable inert plastics are usable, in particular as sheet material, as material for the base plate.

Next, the air vent 7 and the punched hole 24 are punched. A special punching method is employed for the punched hole 24 to enable punching of both the straight edge face 18 and the obliquely extending edge face 19 in one single step. For this purpose, use is made of a two-step knife which first punches the punched hole 24 with straight edge faces by means of a first knife section and subsequently produces the oblique edge face 19 in the lower part 2 by means of a second knife section. In the simplified form of the protection against using the sensor upside-down, which form has straight edges which are not in exact alignment with each other, the punched hole 24 needs to be punched asymmetrically, e. g. is slightly offset to the folding line 4, so that the edge face 19a is slightly shifted relative to the edge face 18 with regard to the symmetry to the folding line 4. The special punching method using a two-step knife may then be dispensed with.

After the punching operation, a conducting structure is applied by means of a screen printing method known per se, said conducting structure consisting of the electrodes 8 to 11, the contacts 14 as well as of corresponding connection lines. Gold is the material of choice for the conducting structure due to its good contact properties, but use may be made also of graphite, copper, aluminum, silver, platinum, etc.

The lower part 3 has a section 15 on which the contacts 14 come to rest. Said section 15 is designed for insertion into an evaluation device.

Thereafter, an impression is then incorporated in the section 15 or adjacent thereto at the edges of the base plate 1, which impression later serves as the light guiding elements 21 and 22. If light is incident in said light guiding elements 21 or 22 from the evaluation device with the biosensor inserted therein, said elements will emit light having the color of the incident light. This will serve as a message signal for the patient.

Upon applying the conducting structure, the intermediate layer 5 is applied onto the lower part 2 by a screen printing method. First, a varnish layer having a suitable structure is applied. This varnish has a thickness of 2-5 mm and does not cover the region around the slit 20 and the electrodes 8 and 9. The varnish serves as a spacer and adheres directly to the lower part 3. A thin adhesive layer is applied onto the varnish. Said adhesive is a special one which will not harm the enzyme to be applied later. In printing, the adhesive is also structured such that the slit 6 remains exposed from the air vent 7 to the punched hole 24. The intermediate layer 5 formed by the adhesive may be applied onto the upper part 2, too.

At this stage of the procedure, it is possible to interrupt the manufacturing process for almost as long as desired. One merely needs to cover the adhesive of the intermediate layer 5 with a protective sheet. This presents the advantage that the subsequent step, in which an enzyme-containing, in some cases perishable substance is applied, may be effected a relatively short time before the finished sensor can be distributed to consumers. The base plate 1 prepared up to this point may thus be pre-produced in large quantities.

To finish the biosensor, a pasty enzyme-containing substance is applied, in some cases after removal of the protective sheet, onto the electrodes 8, 9 of the lower part 3 or onto the electrodes 10, 11 of the upper part 2. In this case, the degree of viscosity of said substance is set such that the substance does not run between the electrodes and does not mix. Setting the degree of viscosity in this manner allows differently prepared pastes to be applied on the two electrodes. After coating, the pastes have a thickness of 2 to 11 μm.

The base plate 1 is then folded along the folding line 4, whereby the adhesive serving as the intermediate layer 5 bonds the lower part 2 together with the upper part 3. This folding operation is symbolized by the arrow 23 of FIG. 3, wherein the intermediate layer 5 is not shown, however, for the sake of better visibility. The biosensor is thus ready to use.

Now, the biosensor is used as follows.

First, the biosensor is inserted into an evaluation device which electronically connects the contacts 14 and thus establishes a connection to the electrodes 8 to 11.

The patient is then required to puncture his fingertip in order to obtain a drop of blood, for example when the blood sugar level is to be determined. Subsequently, the patient places the finger with the drop of blood in the recess 17. In doing so, he will benefit from the centering function resulting from the design of the edge faces at the supply inlet 16: there is no need for him to move his finger obliquely toward the recess 17, but the edge face, for example the oblique edge face 19, automatically leads the finger toward the supply inlet 16.

The drop of blood is drawn through the capillary channel 20 via the pairs of electrodes 11, 12 and 10, 8 by the capillary forces, which differ in strength according to the design of the capillary channel 20 as a non-straight or oblique capillary channel.

Then, the following process takes place between each pair of electrodes.

The paste applied on one electrode each of the pairs of electrodes contains a substance-specific enzyme, so that a redox reaction will take place wherein the electrons are transferred into a metabolite from the substance to be determined, for example glucose, to the enzyme with conversion of the substance to be determined. Thereafter, the electrodes are transferred from the enzyme contained in the substance to an electrode, which may be facilitated, in some cases, by a known mediator, e.g. ferrocene.

The current generated in this way is measured. Alternatively to the electrocemical measurement, one may also measure conductivity.

The electrochemical development is then continuously observed over a certain period of time, resulting in a curve which may be evaluated with regard to various characteristic quantities, for example in respect of its slope, absolute height, etc. This is known to the person skilled in the art.

According to the reading resulting from the measurement, the evaluation device then indicates the content of the substance to be determined in the blood. In this case, a qualitative indication of the substance to be detected may be effected, e. g. in the sense of a yes/no statement as to whether a limit value has been exceeded; however, a quantitative determination, e.g. by indicating a concentration, is also possible. It is further possible to provide an additional indication, when a limit value to be set is exceeded, or to check the plausibility of the result of measurement.

The biosensor allows several readings to be obtained from one single body liquid sample due to the at least two pairs of electrodes. For this purpose, differently prepared pastes containing different enzymes are to be provided at the pairs of electrodes. Alternatively, it is possible to use a pair of electrodes for a reference measurement. The conditions of the reference measurement correspond to the above-mentioned measurement of blood sugar levels, except that the substance used therein does not contain any enzymes, but otherwise, if possible, has the same properties. The reference measurement may then also be compared with certain limit values, e.g. in order to effect a plausibility check. Further, said reference measurement is evaluated according to the same criteria as the measurement at the pair of electrodes with the enzyme-containing paste, wherein the results of measurement of the reference measurement serve as zero values. This will allow an improvement in the precision of measurement as also known, for example, from the aforementioned EP-A 0 359 831.

In conducting said measurement, the light guiding elements 21, 22 serve to convey information to the patient. For example, the light guiding element 21 in the evaluation device may be coupled to a red LED and the light guiding element 22 may be coupled to a green LED. This allows to indicate to the patient, whether the biosensor is ready for measurement, after it has been inserted into the evaluation device. For example, the light guiding surface 21, which is coupled to the green LED, may be illuminated during that period of time in which the patient can introduce the body liquid into the supply inlet 16. If the evaluation device has recognized that a sufficient volume of body liquid has been applied, the illumination of the light guiding elements 21, 22 may be switched correspondingly so as to indicate to the patient, for example by red light, that a valid measurement has been effected or that the sensor element is not ready to receive further body liquids.

1. A biosensor for determining substances in body liquids, in particular in blood, which biosensor comprises:
an upper part (2) and a lower part (3), lying on top of each other, and
an intermediate layer (5), which is located between the upper part (2) and the lower part (3) and in which a slit (6) is formed, wherein
said upper part (2), said lower part (3) and said slit (6) form a capillary channel (20), which extends from a supply inlet (16) formed at the edge of the biosensor to an air vent (7) formed in the upper or the lower part (2, 3), and
electrodes are provided which, together with an enzyme-containing substance, allow an electrochemical measurement of the substances to be determined, characterized in that
both the upper part (2) and the lower part (3) each carry at least one electrode (8, 9; 10, 11) in the region of the capillary channel (20), said electrodes being arranged in pairs lying opposite each other in the capillary channel (20), and an enzyme-containing substance (12, 13) is applied on at least one electrode (8, 9) of at least one pair of electrodes.

2. The biosensor as claimed in claim 1, characterized in that the upper and lower parts (2, 3) are parts of a base plate (1) which is folded along a folding line (4), on which the upper and lower parts (2, 3) are joined together.

3. The biosensor as claimed in claim 2, characterized in that the electrodes (8, 9; 10, 11) are connected to contacts (14) via conducting strips, said contacts (14) contacting the upper and/or lower part (2, 3) at a section which preferably protrudes from the lower or the upper part (3, 2) and is provided for insertion into an evaluation device.

4. The biosensor as claimed in claim 2 or 3, characterized in that the supply inlet (16) is located on the edge of the biosensor formed by the folding line (4).

5. The biosensor as claimed in any one of the preceding claims, characterized in that the supply inlet (16) is provided in the form of a curved recess (17) in the upper and lower parts (2, 3).

6. The biosensor as claimed in claim 5, characterized in that, in the lower part (3), the recess (17) has an edge face (18) which is located perpendicularly to the top surface of the lower part (3), and in the upper part (2), it has an edge face (19) which is oblique relative to the top surface of the upper part (3).

7. The biosensor as claimed in claim 3, characterized in that the width of the slit (6) in the intermediate layer (5), as seen facing away from the supply inlet (16), increases or decreases, so that the capillary channel (20) is provided as an oblique capillary channel.

8. The biosensor as claimed in claim 3, characterized in that a light guiding element (21, 22) is formed on one edge or on both edges of the upper or the lower part (2, 3) comprising the protruding section (15).

9. The biosensor as claimed in any one of the preceding claims, characterized in that the enzyme in the enzyme-containing substance is selected from the group consisting of lactate oxidase, glucose oxidase, cholesterase, uricase, xanthine oxidase, peroxidase, urease, aminotransferase, cholesterol oxidase, aminooxidase, glutamate oxidase, creatinine oxidase, creatinine aminohydrolase and dehydrogenase.

10. A method of producing a biosensor as claimed in any one of the preceding claims, said method comprising the steps of:
a) producing a base plate, which comprises two parts connected with each other and foldable along a folding line, one of said parts of the base plate forming an upper part and the other of said parts forming a lower part of the biosensor,
b) forming a through hole in the base plate,
c) applying a conducting structure comprising electrodes, which are connected to contacts on the upper or the lower part via conductors, wherein at least one electrode is arranged on the upper part and the same number of electrodes are arranged on the lower part, and
d) applying an intermediate layer onto the upper or the lower part, said intermediate layer being provided with a slit which extends up to the hole and is arranged above the electrodes of the upper or the lower part.

11. The method as claimed in claim 10, characterized in that the following step is carried out after step d):
e) applying an enzyme-containing paste onto each of the electrodes arranged in the slit.

12. The method as claimed in claim 11, characterized in that, following step e), the base plate is folded along the folding line, so that the intermediate layer lies between the upper and lower parts, and that a capillary channel is formed by the slit, the upper part and the lower part, in which capillary channel the electrodes on the upper and lower parts are arranged in pairs lying opposite each other.

13. The method as claimed in any one of the preceding method claims, characterized in that, preferably prior to step c), a preferably lens-shaped hole is punched in the base plate in the region of the folding line, said hole being formed such that, during folding, the edge of the hole lying in the lower part comes to rest in the region of the edge of the hole lying in the upper part and that the slit is formed so as to extend to said hole.

14. The method as claimed in claim 13, characterized in that, when the hole is being punched, the edge of the hole lying in the lower part obtains a vertical edge face and the edge of the hole lying in the upper part obtains an oblique edge face, and that, during folding, the edge of the hole lying in the lower part comes to rest on the edge of the hole lying in the upper part.

15. Use of the biosensor as claimed in any one of preceding claims 1 to 9 for determining at least one blood parameter, preferably selected from the group: blood sugar level, urea, lactate, cholesterol, vitamins, troponin and myoglobin.
